(19) United States
(12) Patent Application Publication
(10) Pub. No.: US 2026/0102293 A1
(43) Pub. Date: Apr. 16, 2026

(54) SPIDERWEB CORE DESIGNED DISPOSABLE DIAPERS

(71) Applicant: Mame Diarra Tongia Monique Sylla, Broklyn, NY (US)

(72) Inventor: Mame Diarra Tongia Monique Sylla, Broklyn, NY (US)

(21) Appl. No.: 19/296,460

(22) Filed: Aug. 11, 2025

Related U.S. Application Data
(60) Provisional application No. 63/681,788, filed on Oct. 11, 2024.

(51) Int. Cl. A61F 13/538; A61F 13/53; A61F 13/56

(52) U.S. Cl. CPC A61F 13/538; A61F 13/5638; A61F 2013/530182; A61F 2013/530364

(57) ABSTRACT

A diaper includes a liquid-pervious top-sheet; a liquid-impervious back-sheet; and a liquid-absorbent spiderweb core between the liquid-pervious top-sheet and the liquid-impervious back-sheet, wherein the liquid-absorbent spiderweb core comprises a plurality of radial lines radiating outward from a central point, and wherein the liquid-absorbent spiderweb core is outward-extendable omni-directionally.

### RELATED APPLICATIONS

The present application claims the benefit of U.S. Provisional Patent Application No. 63/681,788, filed Aug. 10, 2024, entitled “TRUE-TONE FLESH COLOR EXPANDABLE POSTERIOR POCKET DIAPERS AND SANITARY NAPKIN,” which is incorporated by reference herein.

### TECHNICAL FIELD

Aspects and implementations of the present disclosure relate to wearing articles, specifically spiderweb core designed disposable diapers.

### BACKGROUND

Disposable diapers are typically constructed with a waist opening and two leg openings to allow for a secure fit around the wearer's body. However, despite their intended function to absorb and contain body waste, some disposable diapers often experience leakage, particularly around the leg openings. This leakage can occur due to inadequate sealing, poor fit, or insufficient absorbent material distribution near the leg cuffs, leading to discomfort and potential skin irritation for the wearer.

### SUMMARY

The below summary is a simplified summary of the disclosure in order to provide a basic understanding of some aspects of the disclosure. This summary is not an extensive overview of the disclosure. It is intended neither to identify key or critical elements of the disclosure, nor delineate any scope of the particular implementations of the disclosure or any scope of the claims. Its sole purpose is to present some concepts of the disclosure in a simplified form as a prelude to the more detailed description that is presented later.

A diaper is disclosed. The diaper has a front waist region, a rear waist region and a connection region extending between the front waist region and the rear waist region. The diaper includes a liquid-pervious top-sheet, a liquid-impervious back-sheet, and a liquid-absorbent spiderweb core between the liquid-pervious top-sheet and the liquid-impervious back-sheet in the connection region, and the liquid-absorbent spiderweb core comprises a web radiating outward from a central point.

In some embodiments, the liquid-absorbent spiderweb core is in a circular or oval shape. In some embodiments, the web comprises a plurality of radial lines that extend outward and anchor the spiderweb core to surrounding regions of the diaper. In some embodiments, the plurality of radial lines are formed from a bamboo material. In some embodiments, the count of the plurality of radial lines ranges from 10 to 40. In some embodiments, the web is outward-extendable. In some embodiments, the liquid-absorbent spiderweb core is formed from one or more hypoallergenic materials. In some embodiments, the liquid-absorbent spiderweb core is formed from one or more biodegradable materials. In some embodiments, the liquid-absorbent spiderweb core is sandwiched between the liquid-pervious top-sheet and the liquid-impervious back-sheet and attached to at least one of the liquid-pervious top-sheet and the liquid-impervious back-sheet. In some embodiments, the diaper further includes two pairs of end flaps extending in a transverse direction outside longitudinally opposite ends of the liquid-absorbent spiderweb core, and tape fasteners attached to the end flaps in the rear waist region to extend in the transverse direction, and the diaper is adapted to be worn with a pad laid on the liquid-pervious top-sheet and with the tape fasteners being anchored on an outer surface of the front waist region to connect the front and rear waist regions with each other.

An open-type disposable wearing article is disclosed. The open-type disposable wearing article has a front waist region, a rear waist region and a connection region extending between the front waist region and the rear waist region. The open-type disposable wearing article includes a top-sheet, a back-sheet; and a spiderweb core between the top-sheet and the back-sheet in the connection region, and the spiderweb core comprises a web radiating outward from a central point, wherein the spiderweb core is liquid-absorbent, and wherein the web is outward-extendable.

In some embodiments, the top-sheet is liquid-pervious, and the back-sheet is liquid-impervious. In some embodiments, the spiderweb core is in a circular or oval shape. In some embodiments, the web comprises a plurality of radial lines that extend outward and anchor the spiderweb core to surrounding regions of the diaper. In some embodiments, the plurality of radial lines are formed from a bamboo material. In some embodiments, the count of the plurality of radial lines ranges from 10 to 40. In some embodiments, the spiderweb core is formed from one or more hypoallergenic materials. In some embodiments, the spiderweb core is formed from one or more biodegradable materials. In some embodiments, the spiderweb core is sandwiched between the top-sheet and the back-sheet and attached to at least one of the top-sheet and the back-sheet. In some embodiments, the open-type disposable wearing article further includes two pairs of end flaps extending in a transverse direction outside longitudinally opposite ends of the spiderweb core, and tape fasteners attached to the end flaps in the rear waist region to extend in the transverse direction, and the diaper is adapted to be worn with a pad laid on the top-sheet and with the tape fasteners being anchored on an outer surface of the front waist region to connect the front and rear waist regions with each other.

Optional features of one aspect may be combined with other aspects where appropriate.

### BRIEF DESCRIPTION OF THE DRAWINGS

Aspects and implementations of the present disclosure will be understood more fully from the detailed description given below and from the accompanying drawings of various aspects and implementations of the disclosure, which, however, should not be taken to limit the disclosure to the specific aspects or implementations, but are for explanation and understanding only.

FIGS. 1A-1C are schemes illustrating an example disposable diaper with a spiderweb core, in accordance with implementations of the present disclosure.

FIG. 2 illustrates an example of a spiderweb core of a disposable diaper, in accordance with implementations of the present disclosure.

FIGS. 3A-3C illustrate examples of disposable diapers with spiderweb cores, in accordance with implementations of the present disclosure.

FIGS. 4A-4D illustrate an example disposable diaper with a spiderweb core in a closed state, in accordance with implementations of the present disclosure.

FIGS. 5A-5D illustrate an example disposable diaper with a spiderweb core in an open state, in accordance with implementations of the present disclosure.

FIGS. 6A-6B illustrate an example disposable diaper with a spiderweb core in various states, in accordance with implementations of the present disclosure.

### DETAILED DESCRIPTION

Disposable diapers are personal hygiene products engineered to absorb and retain body waste, primarily for infants and toddlers, but also for adults experiencing incontinence. A typical disposable diaper may include a front waist region, a rear waist region, and a crotch or connection region extending between these waist regions. The connection region generally consists of multiple functional layers, including a liquid-permeable top sheet (which contacts the user's skin and allows fluid to pass through), an absorbent core (which captures and retains fluids), and a liquid-impermeable back sheet (which prevents leakage to the outside of the diaper). These components are designed to work synergistically to keep the user's skin dry, minimize leakage, and provide comfort and flexibility during use.

Advancements in disposable diaper technology have focused on improving absorption efficiency, reducing overall bulk, enhancing ergonomic fit, and promoting skin health through the use of breathable and hypoallergenic materials. Despite these improvements, several challenges persist. Users and caregivers frequently encounter issues such as leakage during extended wear, discomfort due to inadequate fit or chafing, and insufficient breathability, which can lead to skin irritation or rashes. Additionally, the environmental impact of single-use diapers, which contribute significantly to landfill waste, has prompted a demand for more sustainable materials and innovative designs that reduce ecological footprint while maintaining or improving product performance.

Aspects of the present disclosure address at least the aforementioned drawbacks by providing a novel disposable diaper configuration featuring a spiderweb-designed liquid-absorbent core, also referred to as a liquid-absorbent spiderweb core (or simply spiderweb core), that overcomes existing limitations and offers enhanced functionality for users and caregivers alike. This spiderweb core is positioned between a liquid-pervious top sheet and a liquid-impervious back sheet. The spiderweb core includes a web structure radiating outward from a central point, designed to efficiently distribute and retain liquid throughout the absorbent area. The web structure is outward-extendable and includes radial lines that extend from the center and anchor the spiderweb core to surrounding regions of the diaper, thereby improving structural integrity and fluid management. These radial lines may be constructed from bamboo fibers, which offer both strength and biodegradability. The spiderweb core itself may be fabricated from one or more hypoallergenic and/or biodegradable materials, such as plant-based superabsorbent polymers, cellulose fibers, or other sustainable absorbent materials. This configuration not only enhances fluid distribution and absorption capacity but also addresses environmental concerns by utilizing renewable and compostable materials wherever possible.

In some implementations, the spiderweb pattern may be visible on both the interior and exterior surfaces, and can be engineered with concentric and radial elastic segments. These segments can expand omnidirectionally, including upward, downward, diagonally, and across the interior surface, allowing the diaper to dynamically fit to the body's anatomy regardless of position or motion of the body. The radial geometry ensures equal distribution of stretch force, enhancing both comfort and functionality as the body moves or sits.

The spiderweb-designed diaper integrates a multi-directional expansion system that distinguishes it from conventional diapers, which typically expand only along the horizontal (left-to-right) axis. The spiderweb-designed diaper features an integrated spiderweb stretch pattern that enables adaptive expansion and contraction, while preserving targeted containment zones to prevent leaks and blowouts.

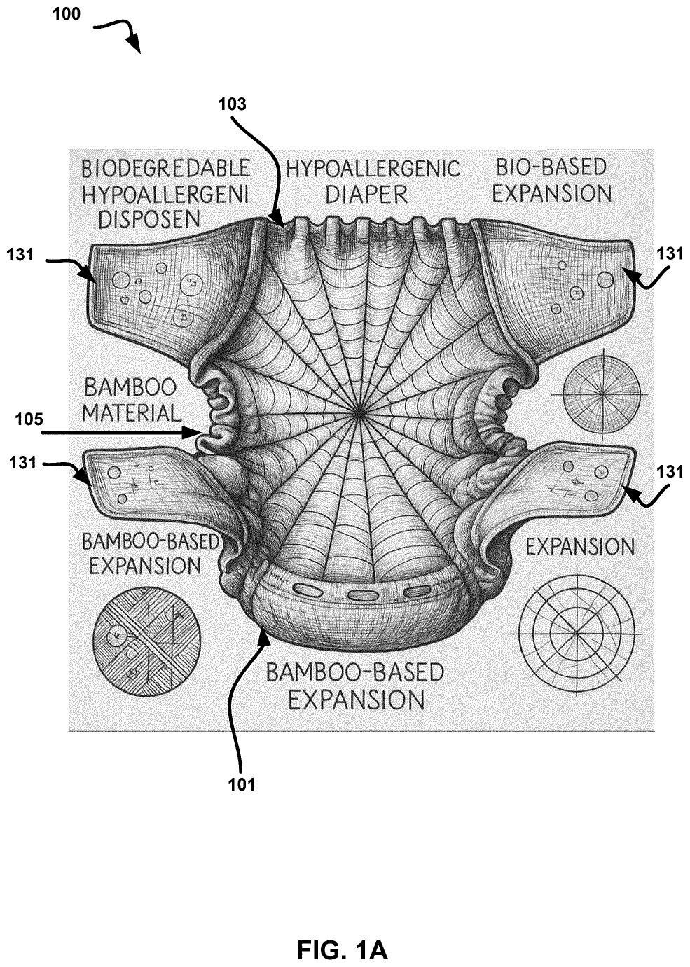

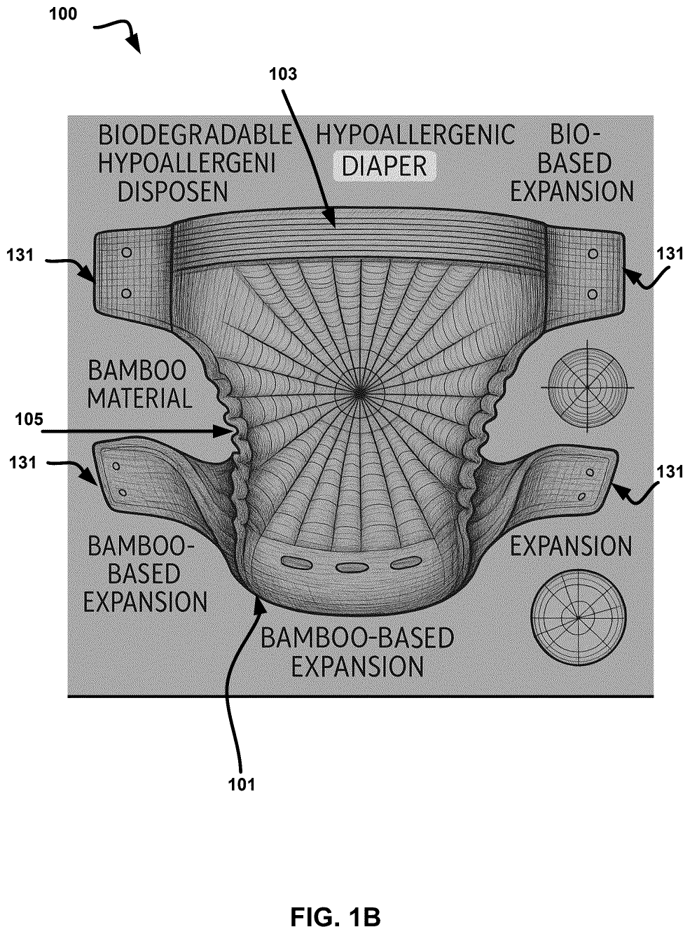

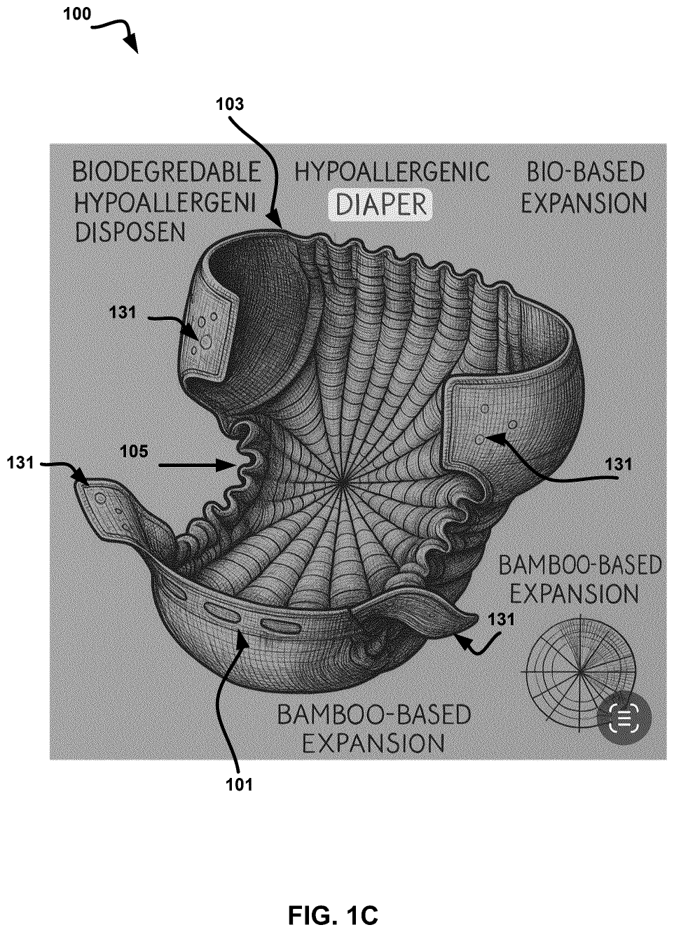

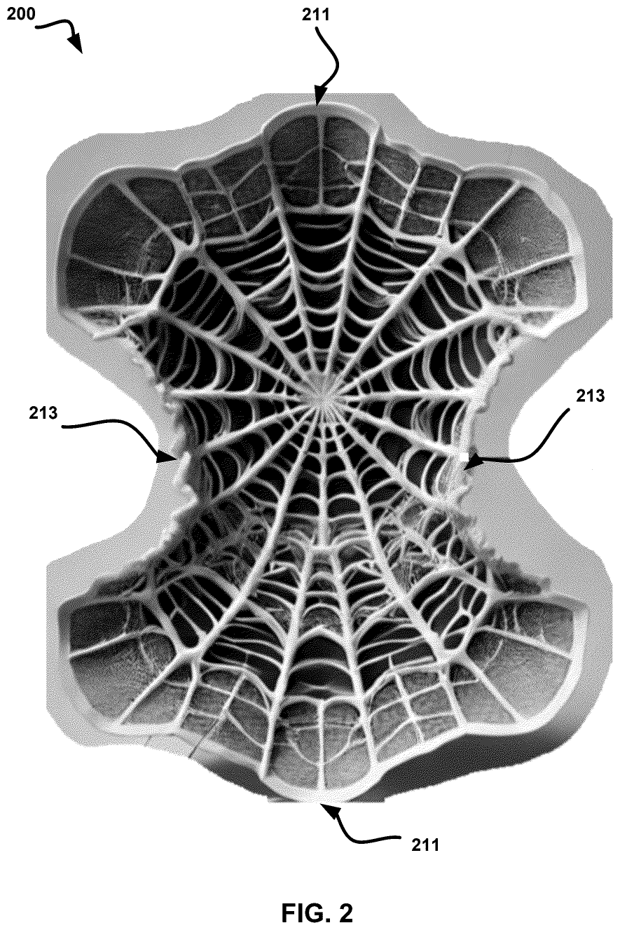

FIGS. 1A-1C illustrate an example disposable diaper (“diaper”) 100, in accordance with implementations of the present disclosure. FIG. 1A illustrates a front elevation view of the disposable diaper in the open state. FIG. 1B illustrates a back elevation view of the disposable diaper in the open state. FIG. 1C illustrates a perspective view of the disposable diaper in the open state. FIG. 2 illustrates an example of the detailed spiderweb of a spiderweb-designed disposable diaper, in accordance with implementations of the present disclosure. Referring to FIGS. 1A-1C and 2, the diaper 100 includes a front waist region 101, a rear waist region 103, and a connection region 105 extending between the front waist region 101 and the rear waist region 103. Each of the front waist region 101 and the rear waist region 103 may include one or more waistbands. The spiderweb-designed pattern may be visually discernible on the front and rear, indicating the presence of omnidirectional expansion capability embedded within the fabric structure. The diaper 100 is in wearable condition, with the waist panel in its neutral height and elastic cuffs for the leg cavities remaining minimally expanded to ensure leak protection.

The diaper 100 may include a top-sheet, a back-sheet, and a liquid-absorbent spiderweb core (“spiderweb core”) 200. The spiderweb core 200 may have a given thickness and be interposed between the top-sheet and the back-sheet. The spiderweb core 200 may be in the connection region and extend between the front waist region and the rear waist region. The spiderweb core 200 may have longitudinally opposite ends 211 of the core 200 and transversely opposite side edges 213 of the core 200. In the connection region 105, the opposite ends 211 curve inward as viewed in the longitudinal direction of the diaper, and the side edges 213 curve inward as viewed in the transverse direction of the diaper.

The spiderweb core 200 may be a web in a circular or oval shape, radiating outward from a central point like the spokes of a wheel. This central point can be the part used most heavily to absorb the liquid. From the central point, long radial lines (e.g., 10 to 40 lines) extend outward and anchor the spiderweb core to surrounding regions. These radial lines support the spiral threads that coil outward from the center in a logarithmic spiral, and the spiral begins tightly wound in the center and becomes more widely spaced as it moves outward.

The radial lines of the spiderweb core 200 may be formed from the bamboo material. The other part of the spiderweb core 200 may be formed from one or more liquid-absorbent materials. The liquid-absorbent materials may include superabsorbent polymers, fluff pulp fibers, thermoplastic synthetic resin fibers, or a mixture thereof. The spiderweb core 200 is bonded to the respective inner surfaces of the top-sheet and the back-sheet. The spiderweb core 200 may be formed from one or more hypoallergenic materials (e.g., organic cotton, bamboo fabric, etc.). The spiderweb core 200 may be formed from biodegradable materials (e.g., cotton, wool, etc.).

The top-sheet is formed from one or more liquid-pervious materials (e.g., hydrophilic fibrous nonwoven fabric). The back-sheet is formed from one or more liquid-impervious materials (e.g., a hydrophobic fibrous nonwoven fabric and a breathable liquid-impervious plastic film laminated together).

The diaper 100 is formed with two pairs of end flaps 131 extending in the transverse direction outside longitudinally opposite ends 211 of the core 200. The end flaps 131 are provided with a pair of liquid-impervious leak-barrier sheets (e.g., formed from a hydrophobic fibrous nonwoven fabric) extending in the longitudinal direction so as to be spaced from and opposed to each other and provided with tape fasteners (e.g., tape fasteners 433) extending in the transverse direction. Each of the tape fasteners has a fixed end and a free end. Each of the tape fasteners is wide enough to provide a secure joint between the connecting components.

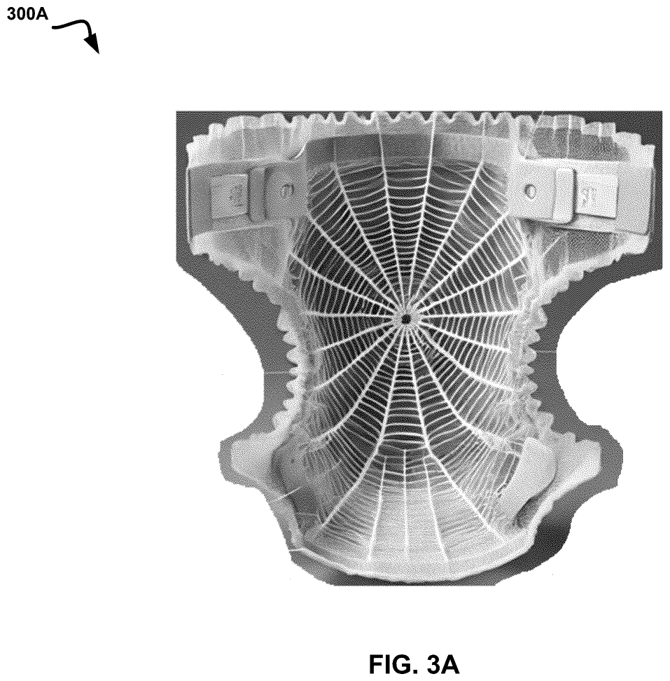

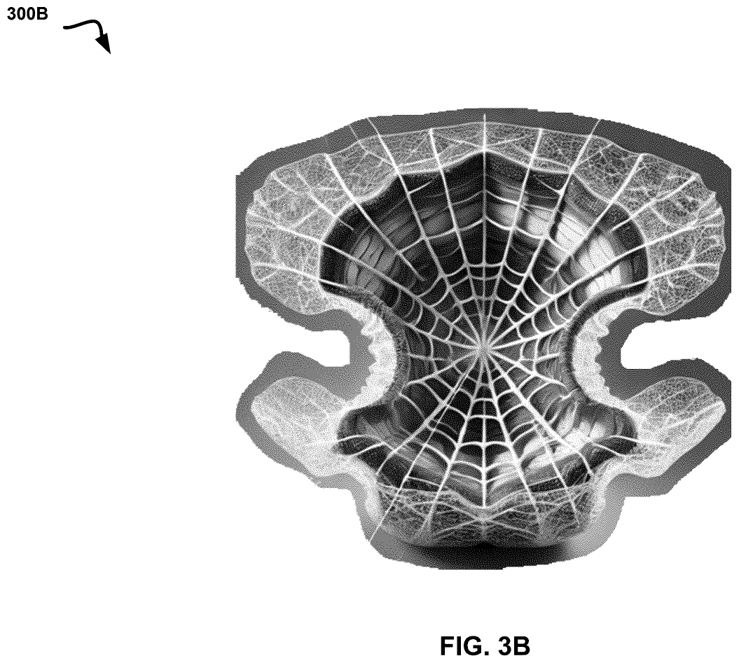

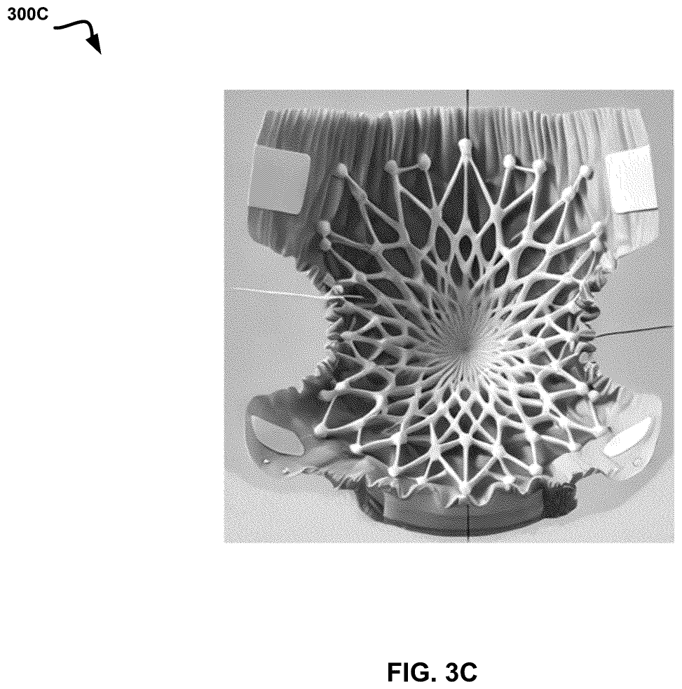

The diaper 100 is disposable and can be hypoallergenic and/or biodegradable. The diaper 100 may be formed from one or more hypoallergenic materials (e.g., organic cotton, bamboo fabric, etc.). The diaper 100 may be formed from one or more biodegradable materials (e.g., cotton, wool, etc.). FIGS. 3A-3C illustrate examples of disposable diapers 300A-300C that include spiderweb cores with various design details, in accordance with implementations of the present disclosure.

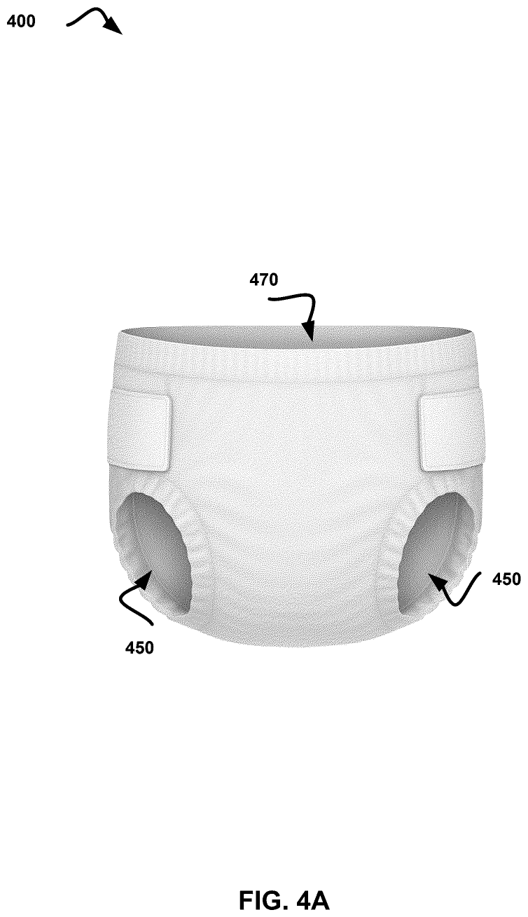

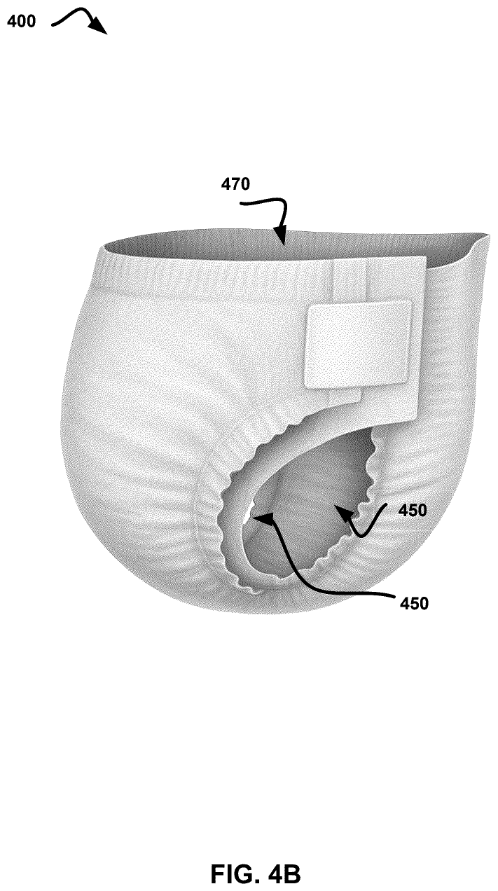

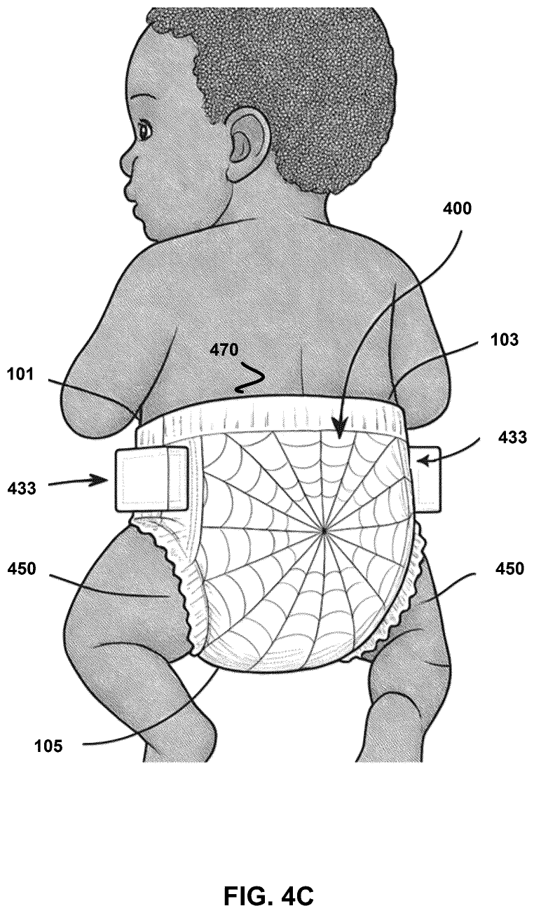

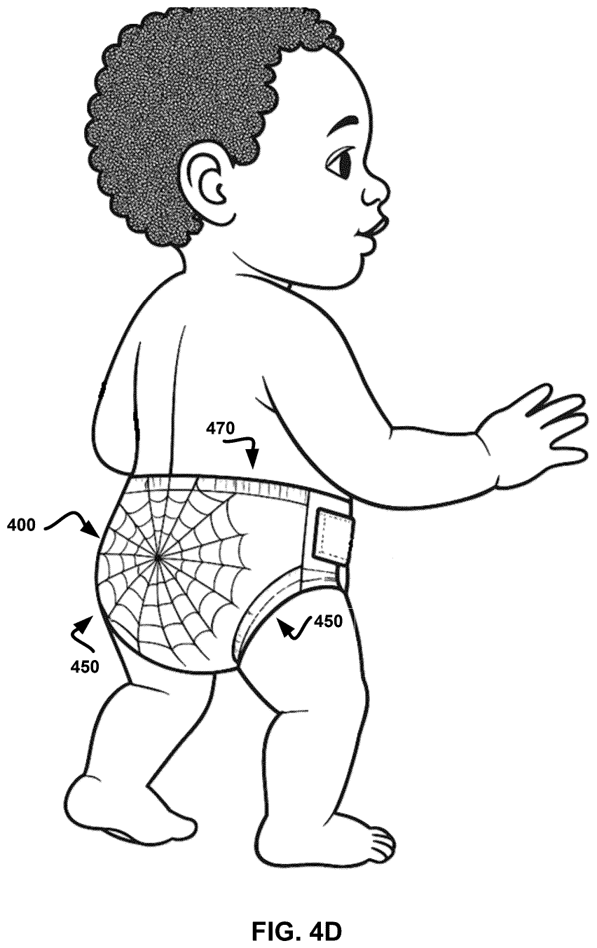

In the course of wearing the diaper (i.e., in the closed state), the diaper 100 can form a top cavity (e.g., waist cavity 470) for wrapping around the waist and two side cavities (e.g., leg cavities 450) each one for wrapping around a proximal thigh of a leg. FIGS. 4A-4D illustrate an example disposable diaper 400 in the closed state, in accordance with implementations of the present disclosure. FIG. 4A illustrates a front elevation view of the disposable diaper in the closed state. FIG. 4B illustrates a side elevation view of the disposable diaper in the closed state. FIGS. 4C and 4D illustrate a human being wearing the disposable diaper (i.e., in the closed state). The disposable diaper with the spiderweb core would provide a natural fit on the body of the human being. In some implementations, the spiderweb core is extensible toward the rear waist region 103 more than the front waist region 101. In some implementations, the connection region 105 include a front potion and a rear potion, and the spiderweb core is placed in the rear potion of the connection region 105. The waistband and hip zones feature spiderweb radial stretch zones, designed to contour naturally around a range of waist sizes while maintaining a snug, skin-safe fit. The tape fasteners 433 do not rely solely on lateral movement; instead, they flex outward and upward, adapting to growth spurts or movement while reducing gapping.

The disposable diaper 400 expands in the vertical (waist-to-crotch) axis and expands with increased crotch pocket volume to accommodate stool or urine without compressing the waste against the skin. This crotch pocket may be enabled by elasticized gussets that are integrated with the spiderweb architecture.

The leg cavities 450 may be reinforced with minimal-stretch elastic cuffs. The leg cavities 450 may be strategically restricted to reduce expansion beyond a calibrated threshold. This ensures a tight seal around the thighs, creating a leak-proof barrier under intensive movement conditions (e.g., crawling, squatting, running). This limited expansion zone reduces blowouts by channeling waste upward into the absorbent core rather than escaping through the leg cuffs. The waist cavity 470 may be engineered for adaptive flex and breathability.

The disposable diaper 400 may be in a fully expanded configuration, illustrating the maximum stretch capacity of the spiderweb core. The waistband is depicted at its full vertical and circumferential expansion, while the rear and front waist regions are visibly pulled outward and upward to create an enlarged volume retention area. The leg cuffs are shown taut, maintaining closure to prevent leakage. The inner spiderweb core and outer shell may expand in a radial pattern, avoiding sagging or distortion. The disposable diaper 400 provides the omni-directional and volumetric adaptability of a diaper and contrasts with traditional diapers that expand primarily in a lateral direction.

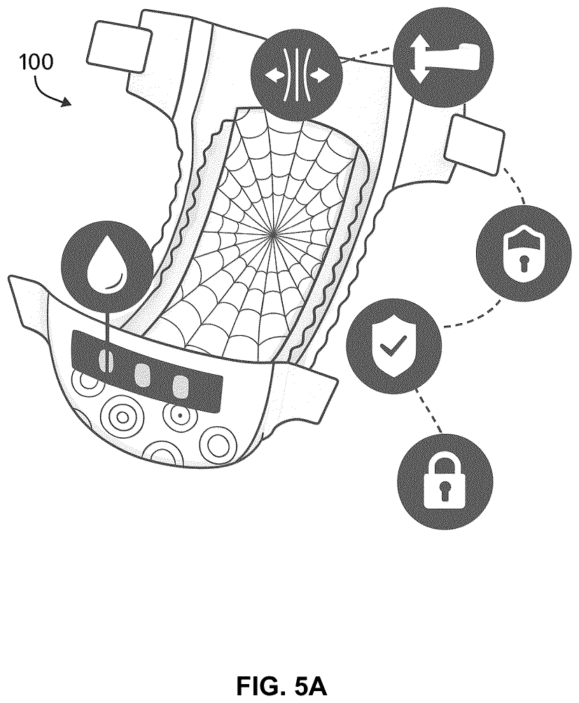

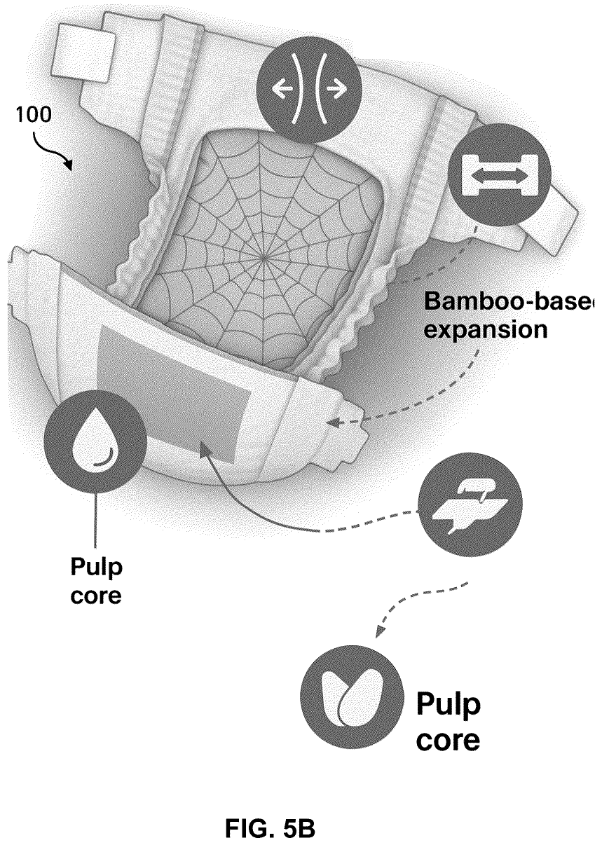

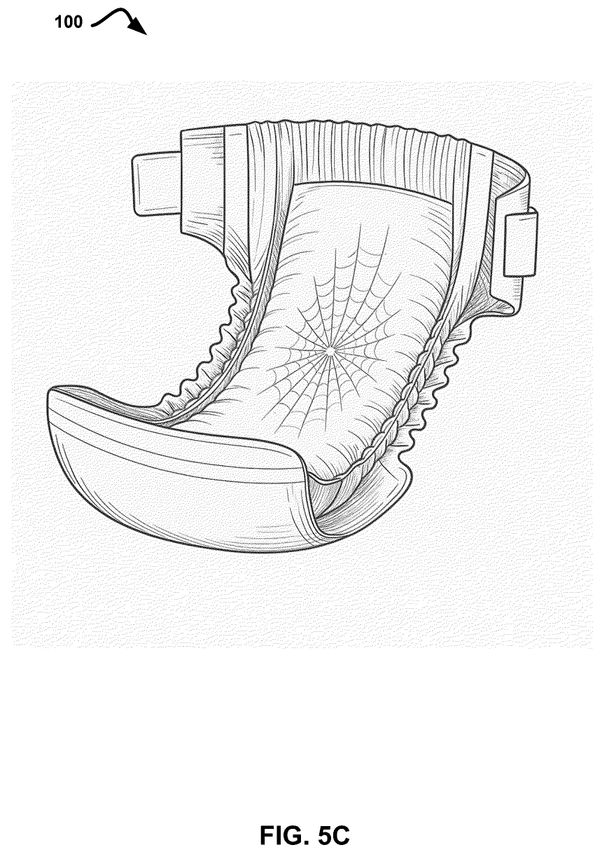

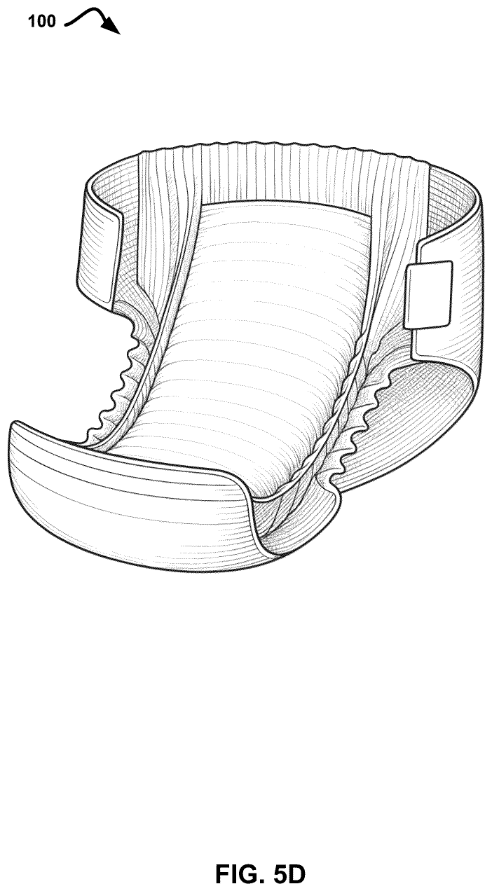

In an open state, the diaper 100 can be flat with inward curving. FIGS. 5A-5D illustrate an example disposable diaper 100 in the open state with various design details, in accordance with implementations of the present disclosure.

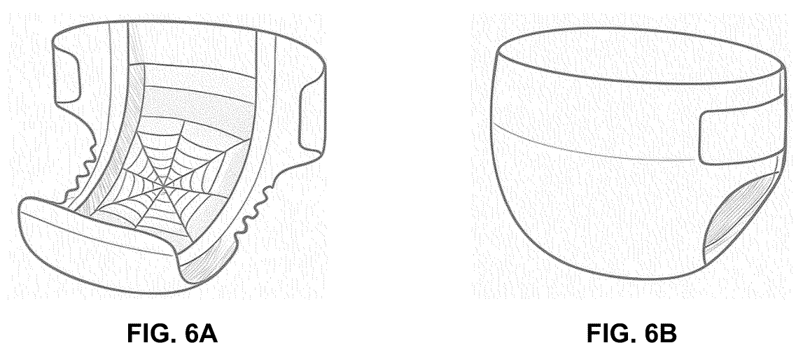

FIGS. 6A-6B illustrate an example disposable diaper with a spiderweb core in various states, in accordance with implementations of the present disclosure. FIG. 6A illustrates a disposable diaper in a contracted, at-rest configuration, illustrating the open state of the diaper with the spiderweb core unengaged. FIG. 6B illustrates a disposable diaper in a stretched configuration, illustrating the closed state of the diaper with the spiderweb core extending from the center of the inner core outward to the front, back, and lateral side panels.

Reference throughout this specification to “one implementation,” or “an implementation,” means that a particular feature, structure, or characteristic described in connection with the implementation is included in at least one implementation. Thus, the appearances of the phrase “in one implementation,” or “in an implementation,” in various places throughout this specification can, but are not necessarily, referring to the same implementation, depending on the circumstances. Furthermore, the particular features, structures, or characteristics may be combined in any suitable manner in one or more implementations.

To the extent that the terms “includes,” “including,” “has,” “contains,” variants thereof, and other similar words are used in either the detailed description or the claims, these terms are intended to be inclusive in a manner similar to the term “comprising” as an open transition word without precluding any additional or other elements.

Moreover, the words “example” or “exemplary” are used herein to mean serving as an example, instance, or illustration. Any aspect or design described herein as “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

It is to be understood that the above description is intended to be illustrative, and not restrictive. Many other embodiments will be apparent to those of skill in the art upon reading and understanding the above description. The scope of the disclosure should, therefore, be determined with reference to the appended claims, along with the full scope of equivalents to which such claims are entitled.

What is claimed is:

1. A diaper comprising:
a liquid-pervious top-sheet;
a liquid-impervious back-sheet; and
a liquid-absorbent spiderweb core between the liquid-pervious top-sheet and the liquid-impervious back-sheet, wherein the liquid-absorbent spiderweb core comprises a plurality of radial lines radiating outward from a central point, and wherein the liquid-absorbent spiderweb core is outward-extendable omni-directionally.

2. The diaper of claim 1, wherein the liquid-absorbent spiderweb core is in a circular or oval shape.

3. The diaper of claim 1, wherein the plurality of radial lines anchor the liquid-absorbent spiderweb core to surrounding regions in the diaper, and wherein the liquid-absorbent spiderweb core and the surrounding regions are configured to channel liquid and waste toward a containment zone formed by the diaper.

4. The diaper of claim 1, wherein the plurality of radial lines are formed from a bamboo material.

5. The diaper of claim 1, wherein a count of the plurality of radial lines ranges from 10 to 40.

6. The diaper of claim 1, wherein the liquid-absorbent spiderweb core comprises a plurality of elasticized connection points configured to allow adaptive sizing.

7. The diaper of claim 1, wherein the liquid-absorbent spiderweb core is formed from one or more hypoallergenic materials.

8. The diaper of claim 1, wherein the liquid-absorbent spiderweb core is formed from one or more biodegradable materials.

9. The diaper of claim 1, wherein the liquid-absorbent spiderweb core is sandwiched between the liquid-pervious top-sheet and the liquid-impervious back-sheet and attached to at least one of the liquid-pervious top-sheet and the liquid-impervious back-sheet.

10. The diaper of claim 1, the diaper further comprising:
two pairs of end flaps extending in a transverse direction outside longitudinally opposite ends of the liquid-absorbent spiderweb core; and
tape fasteners attached to the end flaps in a rear waist region to extend in the transverse direction, wherein the diaper is adapted to be worn with a pad laid on the liquid-pervious top-sheet and with the tape fasteners being anchored on an outer surface of a front waist region to connect the front and rear waist regions with each other.

11. An open-type disposable wearing article comprising:
a top-sheet;
a back-sheet; and
a spiderweb core between the top-sheet and the back-sheet, wherein the spiderweb core comprises a plurality of radial lines radiating outward from a central point, wherein the spiderweb core is liquid-absorbent, and wherein the spiderweb core is outward-extendable omni-directionally.

12. The open-type disposable wearing article of claim 11, wherein the top-sheet is liquid-pervious, and the back-sheet is liquid-impervious.

13. The open-type disposable wearing article of claim 11, wherein the spiderweb core is in a circular or oval shape.

14. The open-type disposable wearing article of claim 11, wherein the plurality of radial lines anchor the spiderweb core to surrounding regions in the open-type disposable wearing article, and wherein the spiderweb core and the surrounding regions are configured to channel liquid and waste toward a containment zone formed by the open-type disposable wearing article.

15. The open-type disposable wearing article of claim 11, wherein the plurality of radial lines are formed from a bamboo material.

16. The open-type disposable wearing article of claim 11, wherein a count of the plurality of radial lines ranges from 10 to 40.

17. The open-type disposable wearing article of claim 11, wherein the spiderweb core is formed from one or more hypoallergenic materials.

18. The open-type disposable wearing article of claim 11, wherein the spiderweb core is formed from one or more biodegradable materials.

19. The open-type disposable wearing article of claim 11, wherein the spiderweb core is sandwiched between the top-sheet and the back-sheet and attached to at least one of the top-sheet and the back-sheet.

20. The open-type disposable wearing article of claim 11, wherein the spiderweb core comprises a plurality of elasticized connection points configured to allow adaptive sizing.
